(19) United States
(12) Patent Application Publication
(10) Pub. No.: US 2013/0053699 A1
(43) Pub. Date: Feb. 28, 2013

(54) APPARATUS AND METHOD FOR PERFORMING PHOTODYNAMIC DIAGNOSIS AND PHOTODYNAMIC THERAPY

(71) Applicants: Wen-Hong Wu, Kaohsiung City (TW); Chung-Hsing Chang, Kaohsiung City (TW); Han-Chao Chang, Hsinchu City (TW); Chun-Li Chang, Puzih City (TW); Kuo-Cheng Huang, Hsinchu City (TW); Din-Ping Tsai, Taipei City (TW); Hsin-Su Yu, Kaohsiung (TW)

(72) Inventors: Wen-Hong Wu, Kaohsiung City (TW); Chung-Hsing Chang, Kaohsiung City (TW); Han-Chao Chang, Hsinchu City (TW); Chun-Li Chang, Puzih City (TW); Kuo-Cheng Huang, Hsinchu City (TW); Din-Ping Tsai, Taipei City (TW); Hsin-Su Yu, Kaohsiung (TW)

(73) Assignees: National Applied Research Laboratories Instrument Technology Research Center, Hsinchu City (TW); Kaohsiung Medical University, Kaohsiung (TW)

(21) Appl. No.: 13/221,145

(22) Filed: Aug. 30, 2011

(51) Int. Cl. A61B 6/00

(52) U.S. Cl. 600/476

(57) ABSTRACT

An apparatus for performing photodynamic diagnosis and photodynamic therapy on a target region that is pre-given with a photosensitizer precursor includes a display unit, an excitation light source operable to irradiate the target region with exciting light so as to excite emission of fluorescence from the target region as a result of fluorescence response of the photosensitizer precursor, an image capturing unit operable to capture a white light image and a fluorescent image of the target region, an image processing unit operable to superimpose the white light image and the fluorescent image into a synthesized image and to provide at least one of the white light image, the fluorescent image and the synthesized image thereto for display on the display unit, and a curing light source operable to irradiate a specified portion of the target region with curing light for treating the specified portion.

### BACKGROUND OR THE INVENTION

1. Field of the Invention

The invention relates to an apparatus and a method for performing photodynamic diagnosis and photodynamic therapy.

2. Description of the Related Art

Photodynamic therapy combines photosensitizer and light for diagnosis or treatment purposes. Conventionally, light with a wavelength in the range between 600 nm and 750 nm is irradiated at a constant intensity onto an area of fixed shape and size (e.g., circular or rectangular shape). However, since the actual area that requires treatment is much smaller than the irradiated area in most situations, a shield is generally required to cover up regions within the irradiated area but not subject to treatment.

To solve the above problem, Taiwanese Patent No. 1283593 discloses an automatic laser displacement control method for laser treatment equipment, which divides a region to be treated into several smaller sub-regions in accordance with the size of a laser light spot. However, the laser is still illuminated at a constant intensity.

In photodynamic diagnosis, or fluorescence diagnosis, a photosensitizer precursor (e.g., 5-ALA) is guided to a region to be treated.

After metabolism, the photosensitizer precursor is excited by ultraviolet (UV) light to generate fluorescence. The generated fluorescence may be captured to form a fluorescent image to facilitate diagnosis by filtering an RGB image to get the red light component with a wavelength ranging between 580 nm to 650 nm. Portions of the region with varying degrees of fluorescence may then be treated differently.

However, at present, multiple independent devices are used for photodynamic therapy and the monitoring of the same without any communication mechanisms in between so that it is necessary for operating personnel to adjust the operating condition/status of the photodynamic therapy based on the monitoring data.

In view of the above, the following drawbacks are present in conventional photodynamic therapy.

1. Since treatment and monitoring of the photodynamic therapy are conducted using separate devices, professional personnel is needed on site to make adjustments to the operating parameters of the treatment in accordance with the monitoring results. Due to differences in personal experiences in the field, individual personnel might make different adjustments under the same circumstance. It is thus difficult to give a general quantized dosage or provide a standardized process for treating diseases.

2. During conventional photodynamic therapy, no mechanism is installed for detecting small dislocations of the region being cured, and thus the position of the laser, which is normally fixed, cannot be adjusted to accommodate such small dislocations.

3. Since the devices are independent and do not communicate with each other, the monitoring results capturing changes in the region being cured is not provided to the devices performing the photodynamic therapy, and therefore the treatment cannot be adjusted in real-time as necessary according to these changes.

### SUMMARY OF THE INVENTION

Therefore, the object of the present invention is to provide an apparatus and a method for performing photodynamic diagnosis and photodynamic therapy that can eliminate the aforesaid drawbacks of the prior art.

According to one aspect of the present invention, there is provided an apparatus for performing photodynamic diagnosis and photodynamic therapy on a target region that is pre-given with a photosensitizer precursor. The apparatus includes a display unit, an excitation light source, an image capturing unit, and image processing unit, and a curing light source.

The excitation light source is operable to irradiate the target region with exciting light having a wavelength which falls within a first range. The target region is excited to emit fluorescence as a result of fluorescence response of the photosensitizer precursor.

The image capturing unit is operable to capture a white light image and a fluorescent image of the target region.

The image processing unit is coupled electrically to the image capturing unit for receiving the white light image and the fluorescent image therefrom, is operable to superimpose the white light image and the fluorescent image into a synthesized image, and is further coupled to the display unit: for providing at least one of the white light image, the fluorescent image and the synthesized image thereto for display on the display unit.

The curing light source is operable to irradiate a specified portion of the target region with curing light having a wavelength which falls within a second range for treating the specified portion. Preferably, the curing light is infrared light.

According to another aspect of the present invention, there is provided a method for photodynamic diagnosis and photodynamic therapy on a target region that is pre-given with a photosensitizer precursor. The method includes the steps of:

(a) irradiating the target region with exciting light having a wavelength which falls within a first range so as to excite emission of fluorescence from the target region as a result of fluorescence response of the photosensitizer precursor;

(b) capturing a white light image of the target region;

(c) capturing a fluorescent image of the target region;

(d) superimposing the white light image and the fluorescent image into a synthesized image;

(e) displaying at least one of the white light image, the fluorescent image and the synthesized image;

(f) defining a specified portion of the target region for treatment; and

(g) irradiating the specified portion of the target region with curing light having a wavelength which falls within a second range.

### BRIEF DESCRIPTION OF THE DRAWINGS

Other features and advantages of the present invention will become apparent in the following detailed description of the preferred embodiments with reference to the accompanying drawings, of which:

FIG. 1 is a schematic block diagram of the first preferred embodiment of an apparatus for performing photodynamic diagnosis and photodynamic therapy on a target region that is pre-given with a photosensitizer precursor according to the present invention;

FIG. 2 is a schematic block diagram of the second preferred embodiment of an apparatus for performing photodynamic diagnosis and photodynamic therapy on a target region that is pre-given with a photosensitizer precursor according to the present invention; and

FIGS. 3a and 3b cooperatively define a flow chart of the preferred embodiment of a method for performing photodynamic diagnosis and photodynamic therapy on a target region that is pre-given with a photosensitizer precursor according to the present invention.

### DETAILED DESCRIPTION OF THE PREFERRED EMBODIMENTS

Before the present invention is described in greater detail, it should be noted that like elements are denoted by the same reference numerals throughout the disclosure.

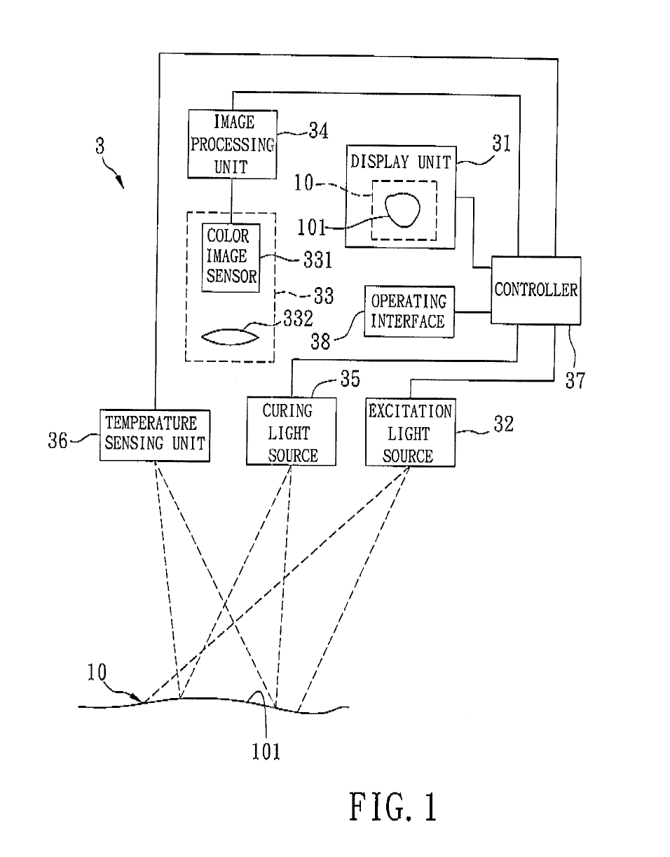

With reference to FIG. 1, the first preferred embodiment of an apparatus 3 according to the present invention is for performing photodynamic diagnosis and photodynamic therapy on a target region 10 that is pre-given with a photosensitizer precursor (not shown) that converts oxygen from air into its toxic form (singlet oxygen) upon irradiation with light falling under a predefined range. Singlet oxygen acts as an intracellular toxin. Preferably, the photosensitizer precursor is 5-Aminolevulinic acid (5-ALA). Due to increased levels of metabolic activity, the topically applied 5-ALA is taken up by cancerous cells most effectively, allowing the cancerous cells to be killed selectively. This means that unaffected tissue remains unharmed and intact while the cancerous tissue around it is being destroyed by the treatment.

The apparatus 3 includes a display unit 31, an excitation light source 32, an image capturing unit 33, an image processing unit 34, a curing light source 35, a temperature sensing unit 36, a controller 37, and an operating interface 38. In this embodiment, the photosensitizer precursor is 5-aminolevulinic acid (5-ALA) or 5-ALA methylesther. However, the present invention is not limited in terms of the photosensitizer precursor used. The target region 10 may be skin of a patient.

The excitation light source 32 is operable to irradiate the target region 10 with exciting light having a wavelength which falls within a first range so as to excite emission of fluorescence from the target region 10 as a result of fluorescence response of the photosensitizer precursor. In this embodiment, the exciting light is ultraviolet (UV) light with a wavelength ranging from 10 nm to 400 nm.

The image capturing unit 33 is operable to capture a white light image and a fluorescent image of the target region 10. In this embodiment, the image capturing unit 33 includes a color image sensor 331 and a first camera lens 332 for focusing light into the color image sensor 331.

The image processing unit 34 this unit also contains a second camera lens is coupled electrically to the color image sensor 331 of the image capturing unit 33 for receiving the white light image and the fluorescent image therefrom, and is operable to superimpose the white light image and the fluorescent image into a synthesized image. The image processing unit 34 is further coupled to the display unit 31 for providing at least one of the white light image, the fluorescent image and the synthesized image thereto for display on the display unit 31.

In this embodiment, the color image sensor 331 captures a color image, which is represented by three primary color light components, i.e., red, green and blue (RGB) light components, with respective intensity values. If the fluorescence emitted by the photosensitizer precursor after being excited by the exciting light is red fluorescence, the fluorescent image is obtained by isolating the red light component of the color image. The image processing unit 34 obtains the synthesized image by performing addition on the RGB light components of the white light image and the fluorescent image.

The curing light source 35 is operable to irradiate a specified portion 101 of the target region 10 with curing light having a wavelength which falls within a second range for treating the specified portion 101. In this embodiment, the curing light source 35 is a laser source. The laser source is capable of performing at least one of pulsed scanning irradiation and continuous irradiation. Moreover, the specified portion 101 of the target region 10 is defined with reference to the synthesized image as a result of superimposing by the image processing unit 34 in accordance with the degree of fluorescence response of the photosensitizer precursor on the target region 10. Specifically, the specified portion 101 is the portion of the target region 10 that exhibits greater fluorescence on the fluorescent image captured by the image capturing unit 33.

The temperature sensing unit 36 is adapted for detecting temperature of the specified portion 101 of the target region 10. In this embodiment, the temperature sensing unit 36 is capable of performing at least one of a point-like temperature detecting and a planar temperature detecting on the specified portion 101 of the target region 10.

The controller 37 is coupled electrically to the curing light source 35 and the temperature sensing unit 36. When the temperature of the specified portion 101 as sensed by the temperature sensing unit 36 exceeds a predefined temperature threshold, the controller 37 deactivates the curing light source 35 to stop irradiation of the specified portion 101 with the curing light. The controller 37 is further coupled electrically to the image processing unit 34. The image processing unit 34 is capable of determining a level of fluorescence corresponding to the specified portion 101 of the target region 10. When the level of fluorescence as determined by the image processing unit 34 is below a predefined fluorescence threshold, the controller 37 deactivates the curing light source 35 to stop irradiation of the specified portion 101 with the curing light.

In this embodiment, the curing light source 35 is movable with respect to the target region 10. The image processing unit 34 is operable to analyze the synthesized image and extract at least one feature of the synthesized image associated with the target region 10. The controller 37 is operable to control movement of the curing light source 35 with reference to said at least one feature in consecutive ones of the synthesized image when the specified portion 101 of the target region 10 is irradiated with the curing light. This is to ensure that the specified portion 101, and not the rest of the target region 10, is treated by the curing light even when there is small dislocation of the target region 10 (e.g., due to movement/dislocation of the patient). It should be noted herein that since the techniques of image tracking and feature extraction are known in the art, further details of the same are omitted herein for the sake of brevity.

The operating interface 38 facilitates user control of intensity and duration of each of the exciting light from the excitation light source 32 and the curing light from the curing light source 35. The user (e.g., doctor, operating technician) may also control the mode of irradiation (pulsed scanning irradiation or continuous irradiation) of the curing light source 35.

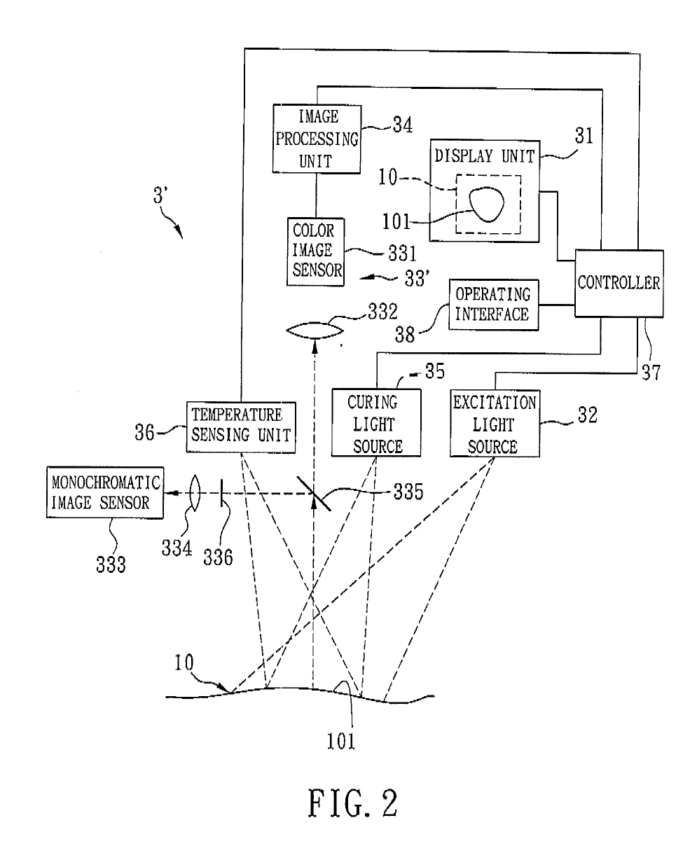

With reference to FIG. 2, the second preferred embodiment of an apparatus 3′ for performing photodynamic diagnosis and photodynamic therapy on a target region 10 that is pre-given with a photosensitizer precursor differs from the first preferred embodiment in that aside from the color image sensor 331 and the first camera lens 332, the image capturing unit 33′ of the second preferred embodiment further includes a monochromatic image sensor 333, a second camera lens 334 for focusing light into the monochromatic image sensor 333, a beam splitter 335 disposed to decompose reflected light from the target region 10 into a first light component that travels toward the color image sensor 331 and a second light component that travels toward the monochromatic image sensor 333, and alight filter 336 disposed between the beam splitter 335 and the monochromatic image sensor 333. In this embodiment, the white light image is captured by the color image sensor 331, while the fluorescent image is captured by the monochromatic image sensor 333. The fluorescent image captured in this manner has better image quality than that of the previous embodiment.

The image processing unit 34 is coupled electrically to both the color image sensor 331 and the monochromatic image sensor 333 of the image capturing unit 33′ for respectively receiving the white light image and the fluorescent image therefrom, and is operable to superimpose the white light image and the fluorescent image into a synthesized image, as with the previous embodiment. In this embodiment, the fluorescence emitted by the photosensitizer precursor after being excited by the exciting light is red fluorescence, and thus the fluorescent image is a red color image.

The present invention will be better understood with reference to the preferred embodiment of a method for performing photodynamic diagnosis and photodynamic therapy on a target region 10 that is pre-given with a photosensitizer precursor.

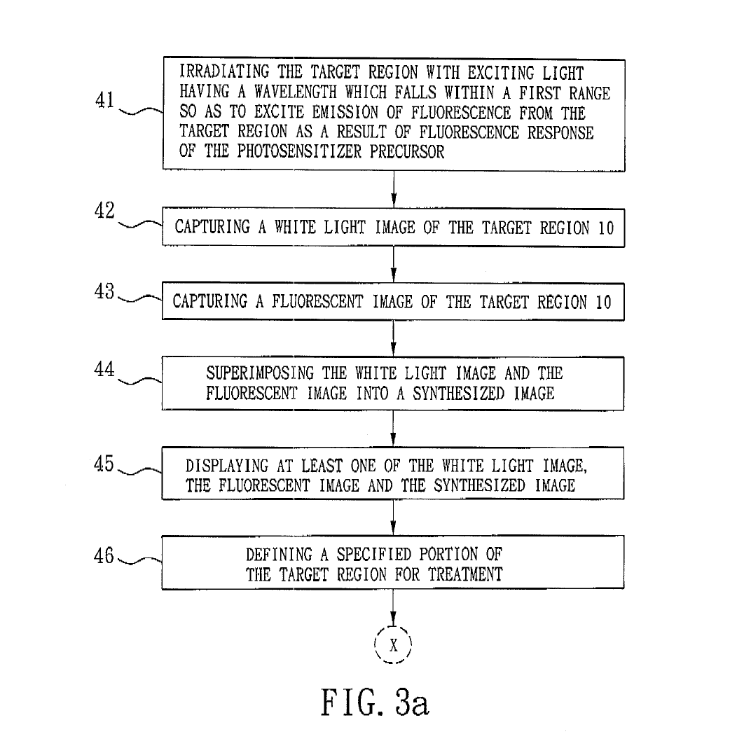

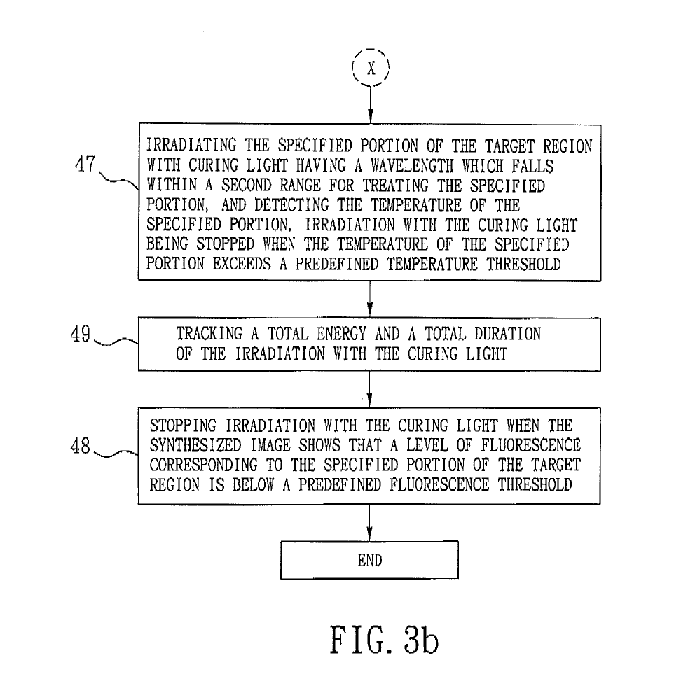

Referring to FIGS. 3a and 3b, the method includes the following steps.

In step 41, the target region 10 is irradiated with exciting light having a wavelength which falls within a first range so as to excite emission of fluorescence from the target region 10 (referring to FIG. 1) as a result of fluorescence response of the photosensitizer precursor. With reference to FIG. 1, this is done by the excitation light source 32, which may be activated to irradiate the exciting light by the controller 37 based on user control, which is inputted via the operating interface 38. In this embodiment, the exciting light is ultraviolet (UV) light.

In step 42, a white light image of the target region 10 is captured. With reference to FIG. 1, the white light image may be captured by the color image sensor 331 of the image capturing unit 33.

In step 43, a fluorescent image of the target region 10 is captured. With reference to FIG. 1, the fluorescent image may also be captured by the color image sensor 331. Alternatively, with reference to FIG. 2, the fluorescent image may be captured by the monochromatic image sensor 333.

It should be noted herein that the present invention is not limited in the order in which steps 42 and 43 are performed.

In step 44, the white light image and the fluorescent image are superimposed into a synthesized image. With reference to FIG. 1, this is performed by the image processing unit 34.

In step 45, at least one of the white light image, the fluorescent image and the synthesized image is displayed.

With reference to FIG. 1, the image to be displayed is transmitted from the image processing unit 34 to the display unit 31 for display on the display unit 31. Preferably, the synthesized image is displayed so as to show both features of the target region 10 that can hardly be seen in the fluorescent image, as well as the fluorescence emitted as a result of fluorescent response of the photosensitizer precursor.

In step 46, a specified portion 101 of the target region 10 is defined for treatment. The specified portion 101 of the target region 10 is defined with reference to the synthesized image in accordance with the degree of fluorescent response of the photosensitive precursor on the target region 10. Specifically, with reference to FIG. 1, with the synthesized image displayed on the display unit 31, the user may define a portion of the target region 10 that exhibits a greater level of fluorescence emission and designate it as the specified portion 101 for subsequent photodynamic treatment.

In step 47, the specified portion 101 of the target region 10 is irradiated with curing light having a wavelength which falls within a second range for treating the specified portion 101. With reference to FIG. 1, the curing light source 35 is used for the irradiation of the curing light. In this embodiment, the curing light is infrared laser. Depending on the actual circumstance, the curing light may be irradiated in a programmed-pulsed scanning manner or a continuous manner.

Preferably, in step 47, the temperature of the specified portion 101 is also detected. The temperature sensing may be a point-like temperature sensing or a planar temperature sensing. Irradiation with the curing light is stopped when the temperature of the specified portion 101 exceeds a predefined temperature threshold (e.g., 40° C.). This decuring measure prevents the specified portion 101 from overheating by the curing light during the photodynamic therapy. Once the temperature of the specified portion 101 drops below the predefined temperature threshold, the irradiation with the curing light may be re-activated.

Optionally, the temperature detected by the temperature sensing unit 36 may be provided to the display unit 31 for display thereon so as to assist the user in determining whether and/or how to adjust the intensity, duration or other parameters of the curing light.

Preferably, steps 42, 43 and 44 are performed periodically to facilitate monitoring the direction of the curing light, and to facilitate monitoring the progress of the photodynamic therapy.

Specifically, since parts of consecutive ones of the synthesized image as attributed to consecutive ones of the white light image can show movement of the target region 10 (e.g., the skin of the patient), the direction of the irradiation of the curing light may be adjusted with reference to the consecutive ones of the synthesized image to ensure that the specified portion 101 of the target region 10 (which demonstrates greater fluorescence response) is being treated. In addition, since parts of the consecutive ones of the synthesized image as attributed to consecutive ones of the fluorescent image can show the progress of the photodynamic therapy on the specified portion 101 in terms of the level of fluorescence corresponding to the specified portion 101, which should diminish as the treatment progresses and takes effect, the intensity and duration of the irradiation of the curing light may be adjusted accordingly. To this end, the method further includes step 48, where irradiation with the curing light is stopped when the synthesized image shows that a level of fluorescence corresponding to the specified portion 101 of the target region 10 is below a predefined fluorescence threshold.

In other words, the white light image is used for monitoring small dislocations of the patient and serves as the basis for adjusting the direction of the irradiation of the curing light, while the fluorescent image is used for monitoring the progress of the treatment and serves as the basis for adjusting the intensity and duration of the irradiation of the curing light.

Preferably, the method further includes step 49 to be performed in between steps 47 and 48, where a total energy and a total duration of the irradiation with the curing light is tracked. The tracking of the total energy and the total duration is completed when the irradiation with the curing light is stopped in step 48. This step facilitates a quantitative recording of the photodynamic treatment for future studies and analysis.

The present invention has the following advantages and effects:

1. With the modular design of the apparatus 3 (3′), modules that are needed or that may influence the photodynamic treatment process can all be integrated. Specifically, since the display unit 31, the excitation light source 32, the image capturing unit 33 (33′), the image processing unit 39, the curing light source 35 and the temperature sensing unit 36 are all integrated into a single apparatus with the controller 37, the photodynamic therapy can be monitored and controlled with the assistance of the information acquired by each module.

2. With the aid of the image capturing unit 33 (33′), the image processing unit 34, and the temperature sensing unit 36, real-time adjustment to the photodynamic therapy is made possible. Specifically, by periodically capturing the white light and fluorescent images of the target region 10 and superimposing them into the synthesized image to monitor the fluorescence response of the photosensitizer precursor in the specified portion 101 of the target region 10 and acquire knowledge of the progress of the photodynamic therapy, and by additionally sensing the temperature of the specified portion 101 to monitor the condition thereof (overheating or not), the direction, intensity and duration of the irradiation with the curing light on the specified portion 101 can be adjusted in real time.

3. The operating conditions/parameters of the photodynamic therapy can be quantified. In addition, differences in the operating condition settings of the photodynamic therapy under the same circumstance as attributed to different operating personnel can be minimized.

In summary, the present invention is capable of obtaining necessary information helpful during photodynamic therapy, and is further capable of using the obtained information to make necessary adjustments to the curing light so as to enhance the photodynamic therapy process.

While the present invention has been described in connection with what are considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. An apparatus for performing photodynamic diagnosis and photodynamic therapy on a target region that is pre-given with a photosensitizer precursor, said apparatus comprising;
a display unit;
an excitation light source operable to irradiate the target region with exciting light having a wavelength which falls within a first range so as to excite emission of fluorescence from the target region as a result of fluorescence response of the photosensitizer precursor;
an image capturing unit operable to capture a white light image and a fluorescent image of the target region;
an image processing unit coupled electrically to said image capturing unit for receiving the white light image and the fluorescent image therefrom, operable to superimpose the white light image and the fluorescent image into a synthesized image, and further coupled to said display unit for providing at least one of the white light image, the fluorescent image and the synthesized image thereto for display on said display unit; and
a curing light source operable to irradiate a specified portion of the target region with curing light having a wavelength which falls within a second range for treating the specified portion.

2. The apparatus as claimed in claim 1, further comprising:
a temperature sensing unit adapted for detecting temperature of the specified portion of the target region; and
a controller coupled electrically to said curing light source and said temperature sensing unit;
wherein, when the temperature of the specified portion as detected by said temperature sensing unit exceeds a predefined temperature threshold, said controller deactivates said curing light source to stop irradiation of the specified portion with the curing light.

3. The apparatus as claimed in claim 2, wherein said temperature sensing unit is capable of performing at least one of a point-like temperature sensing and a planar temperature sensing on the specified portion of the target region.

4. The apparatus as claimed in claim 1, further comprising an operating interface for user control of intensity and duration of each of the exciting light from said excitation light source and the curing light from said curing light source.

5. The apparatus as claimed in claim 1, wherein the exciting light is ultraviolet light.

6. The apparatus as claimed in claim 1, wherein said image capturing unit includes a color image sensor, and a camera lens coupled to said color image sensor and said image processing unit.

7. The apparatus as claimed in claim 6, wherein said image capturing unit further includes a monochromatic image sensor, a beam splitter disposed to split reflected light from the target region into a first light component that travels toward said color image sensor and a second light component that travels toward said monochromatic image sensor, and a light filter disposed between said beam splitter and said monochromatic image sensor.

8. The apparatus as claimed in claim 1, wherein said curing light source is a laser source, and the curing light is laser.

9. The apparatus as claimed in claim 8, wherein said laser source is capable of performing at least one of pulsed scanning irradiation and continuous irradiation.

10. The apparatus as claimed in claim 1, further comprising a controller coupled electrically to said curing light source and said image processing unit;
wherein said image processing unit is capable of determining a level of fluorescence corresponding to the specified portion of the target region; and
wherein when the level of fluorescence as determined by said image processing unit is below a predefined fluorescence threshold, said controller deactivates said curing light source to stop irradiation of the specified portion with the curing light.

11. The apparatus as claimed in claim 1, further comprising a controller coupled electrically to said image processing unit, said curing light source being movable with respect to the target region, said image processing unit being operable to analyze the synthesized image and extract at least one feature of the synthesized image associated with the target region, said controller being operable to control movement of said curing light source with reference to said at least one feature in consecutive ones of the synthesized image when the specified portion of the target region is irradiated with the curing light.

12. The apparatus as claimed in claim 1, wherein the specified portion of the target region is defined with reference to the synthesized image as a result of superimposing by said image processing unit in accordance with the degree of fluorescence response of the photosensitizer precursor on the target region.

13. A method for performing photodynamic diagnosis and photodynamic therapy on a target region that is pre-given with a photosensitizer precursor, said method comprising the steps of:
(a) irradiating the target region with exciting light having a wavelength which falls within a first range so as to excite emission of fluorescence from the target region as a result of fluorescence response of the photosensitizer precursor;
(b) capturing a white light image of the target region;
(c) capturing a fluorescent image of the target region;
(d) synthesizing the white light image and the fluorescent image into a synthesized image;
(e) displaying at least one of the white light image, the fluorescent image and the synthesized image;
(f) defining a specified portion of the target region for treatment; and
(g) irradiating the specified portion of the target region with curing light having a wavelength which falls within a second range for treating the specified portion.

14. The method as claimed in claim 13, wherein temperature of the specified portion of the target region is sensed in step (g), and irradiation with the curing light is stopped when the temperature of the specified portion exceeds a predefined temperature threshold.

15. The method as claimed in claim 13, wherein the exciting light is ultraviolet light and the curing light is infrared laser.

16. The method as claimed in claim 13, wherein, in step (f), the curing light is irradiated in one of a pulsed scanning manner and a continuous manner.

17. The method as claimed in claim 13, wherein steps (b), (c) and (d) are performed periodically, said method further comprising the step of (h) stopping irradiation with the curing light when the synthesized image shows that a level of fluorescence corresponding to the specified portion of the target region is below a predefined fluorescence threshold.

18. The method as claimed in claim 13, wherein the specified portion of the target region is defined with reference to the synthesized image in accordance with the degree of fluorescence response of the photosensitive precursor on the target region.

19. The method as claimed in claim 13, further comprising the step of (i) tracking a total energy and a total duration of the irradiation with the curing light.
